(19) United States
(12) Patent Application Publication
(10) Pub. No.: US 2024/0099573 A1
(43) Pub. Date: Mar. 28, 2024

(54) MINIMALLY INVASIVE CEREBRAL VENTRICULAR ACCESS PROCESSES, PRODUCTS & SYSTEMS FOR DIAGNOSIS, THERAPIES & IMAGING AT ONCE

(71) Applicant: AQUILLO NV, Redlands, CA (US)

(72) Inventor: Charles Emmett Stout, Redlands, CA (US)

(73) Assignee: AQUILLONV, Redlands, CA (US)

(21) Appl. No.: 18/464,321

(22) Filed: Sep. 11, 2023

Related U.S. Application Data
(60) Provisional application No. 63/474,897, filed on Sep. 22, 2022.

(51) Int. Cl. A61B 1/313; A61B 1/00; A61B 1/07; A61B 6/12; A61B 17/34; A61F 2/958; A61M 27/00

(52) U.S. Cl. CPC A61B 1/3137; A61B 1/00154; A61B 1/07; A61B 6/12; A61B 17/3423; A61F 2/958; A61M 27/006; A61M 2025/0166

(57) ABSTRACT

Cerebral Ventricular Access provides a platform for the clinician to addresses hydrocephalus and related cerebral aqueduct and ventricular issues, including CSF drainage and blockage, which may be treated by stenting, alone, and/or in combination with other therapies to outmode the use of craniotomies and shunting for the same and related conditions

### CROSS-REFERENCE TO RELATED APPLICATIONS

This document claims the benefit of priority to U.S. Provisional Application Ser. No. 63/474,765, filed Sep. 12, 2022, and to U.S. Provisional Application Ser. No. 63/474,897 filed Sep. 22, 2022 each of which is hereby incorporated by reference in its entirety.

### CROSS-REFERENCE TO FUNDING SOURCES

None of the subject matters herein are supported by grants or other government research funds.

### BACKGROUND OF THE DISCLOSURES

### Field of the Disclosures

Hydrocephalus has a global prevalence of 85/100,000 individuals, meaning 88/100,000 for pediatrics or 1% of hospital admissions (<+18); further comprising at least about: 11/100,000 for adults (19-64); 175/100,00 for elderly (>=65); 400/100,000 for age>8.

Likewise, Non-communicating (i.e. obstructive) hydrocephalus is result of stenosis or obstruction of the cerebral aqueduct as known to those skilled in the art primary causes of non-communicating hydrocephalus include; Subarachnoid hemorrhage (36% of SAH cases); Head trauma; Congenital; Infection; Surgical complications and CNS tumors—pineal region, inter alia. These and related conditions need to be addressed.

Prior paradigms being replaced include craniotomy and related processes which ostensively engender higher downstream short-term and long-term brain risks along with quality of life issues needing to be supplanted by global healthcare equity reconfiguration strategies, it is respectfully submitted as worthy of Letters patent, as set forth herein, and claimed below.

### EXPRESSLY INCORPORATED BY REFERENCE

The following United States Letters Patents, are expressly incorporated by reference, as if fully set forth herein, to better define the state of neurovascular patent art, without any admission or preclusive status vis-à-vis the instant disclosures, rather-being invented by the instant assignee, they document the evolution of the devices and procedures comprising the Field as further developed below, disclosed, claimed and shown in the subject matters disclosed of the enclosed patent documents incorporated by reference, as if fully set forth herein—U.S. Letters Pat. Nos. 11,768,676; 11,716,518; 11,679,201; 11,744,947; 11,594,314; 11,285,117; 10,960,313; 10,398,887; 10,016,683; 9,808,859; 9,782,084; 9,556,072; 9,023,081; 8,945,712; 8,926,680; 8,585,713; 8,945,143; 8,574,262; 8,545,415; 8,066,757; 8,070,791; 8,197,493; and 8,088,140. The present inventions relate to systems for treating issues in the brain and related vasculature from injury, insult or trauma—however it is respectfully proposed that access tools and uses, while new novel and non-obvious in the back of the brain have often been evolved from or developed based on medical devices and systems often related or secondary to front brain issues, which it is respectfully proposed have counterparts in the back of the brain approach, whereby the other half includes acute conditions related to intracranial bleeding and related insults and injury, both from rupture of aneurysms and attempts to treat them. This includes both (PCHV) Post-hemorrhagic cerebral aneurysm, DCV (see below) and those related conditions from ischemic and hemorrhagic stroke wherein vase-constriction is a modifiable risk factor. Intracranial aneurysms which rupture, and/or those which are treated with traditional endovascular coiling or neurosurgical clipping methodologies often result in the most feared sequelae of subarachnoid hemorrhage, namely Delayed Cerebral Vasospasm (DCV). Unfortunately, prior to the advent of the instant teachings this has not been the case to the extent that practitioners need it to be to achieve optimal clinical outcomes.

### OBJECTS AND SUMMARY OF THE INVENTIONS

Briefly stated, Cerebral Ventricular Access provides a platform for the clinician to addresses hydrocephalus and related cerebral aqueduct and ventricular issues, including CSF drainage and blockage, which may be treated by stenting, alone, and/or in combination with other therapies to outmode the use of craniotomies and shunting for the same and related conditions.

According to embodiments, systems are disclosed including ventricular access modified and specialized Needle Access Systems with improved medical device tools, and modified stenting-means shown and claimed herein, to image and deliver therapy to the ventricular spaces of the brain.

### BRIEF DESCRIPTION OF THE FIGURES

FIG. 1 shows a schematic cartooned depiction of the system of the present invention, whereby the present invention is fully denoued to artisans whereby said subject matters are eligible for United States Letters Patent as new, novel and non-obvious;

FIG. 2 shows a microcatheter endoscopic light source;

FIG. 3 depicts the microwire endoscopic design;

FIG. 4A shows the access port and needle-device for creating access; and

FIG. 4B is an imaged view showing access point and angles to avoid the brain stem with the instant system.

### DETAILED DESCRIPTIONS OF THE PREFERRED EMBODIMENTS OF THE PRESENT INVENTIONS

The present inventors have discovered better ways to, for example, treat ventricular issues related to hydrocephalus—Hydrocephalus is associated with: headache; Nausea/vomiting; Papilledema; Hearing loss; Sleepiness; Gait instability; Urinary incontinenc; Dementia; Coma/death—all without craniotomy.

Likewise, according to the instant teachings—improved delivery is shown.

NOVEL ENHANCED PROCEDURAL Steps, may be performed in the order offered for consideration or variegated based upon other factors developed during the procedure, in all cases including the following critical base elements, as shown and described and claimed below: (i) Access with LP needle and microwire using fluoroscopy; (ii) Remove LP needle and replace with endoscopy microcatheter needle port over the microwire; (iii) Measure intrathecal pressure; (iv) Instill contrast through port for ventriculography; (v) Insert microcatheter endoscopy system into the patient via the port; (vi) Direct catheter over the endoscopic microwire cephalad through the aqueduct under fluoroscopic and endoscopic visualization; (vii) Remove endoscopic microwire and measure 3rd ventricular pressure, and (viii) Perform stent placement (vs balloon angioplasty).

EXCLUSIONARY EMBODIMENTS: Access to ventricular spaces in the brain requires a novel enhanced access path and approach, and it is respectfully proposed may not be achieved by traditional transfemoral access (TFA) or via transradial access (TRA), see, incorporated by reference expressly U.S. Pat. No. 10,398,877, and, US2022/0134061.

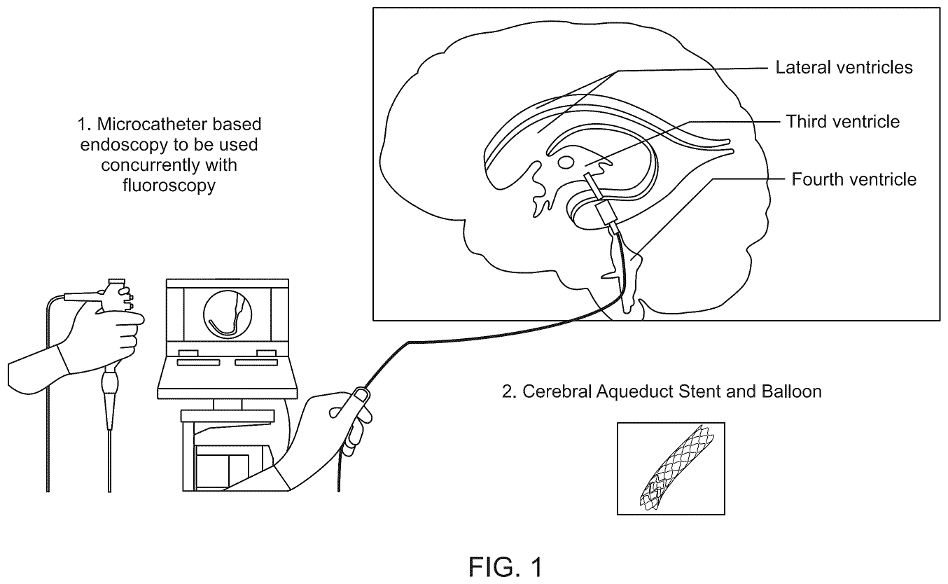

Referring now to FIG. 1, the cartooned schematic illustrates that which is respectfully proposed to be among the Aquillo NV (700 East Redlands Blvd, Suite 714, Redlands, CA 92373) proprietary technology—namely, 1. microcatheter based endoscopy to be used concurrent with fluoroscopy (as known to those of skill in the art) via clinician direction to access a therapy delivery site located in the cerebral aqueduct, illustrated (not to scale) to show an exemplary procedure, relative to the lateral, third and fourth ventricle of a brain; wherein a delivered therapy comprises, 2. at least a cerebral aqueduct stent and balloon.

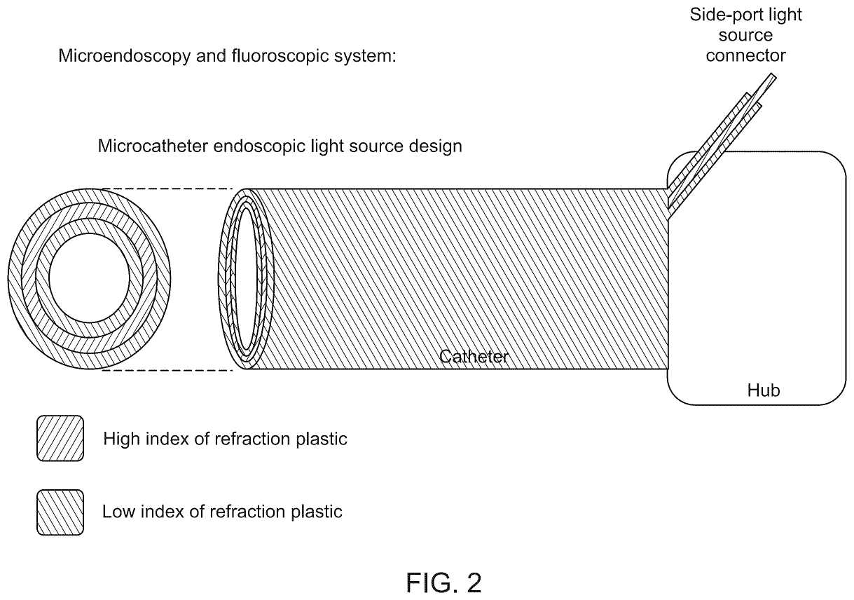

FIG. 2 likewise schematically cartoon illustrates a proposed microendoscopy and fluoroscopic system which includes a cylindrical microcatheter light source design and several performance based characteristics and known to those skilled in the art.

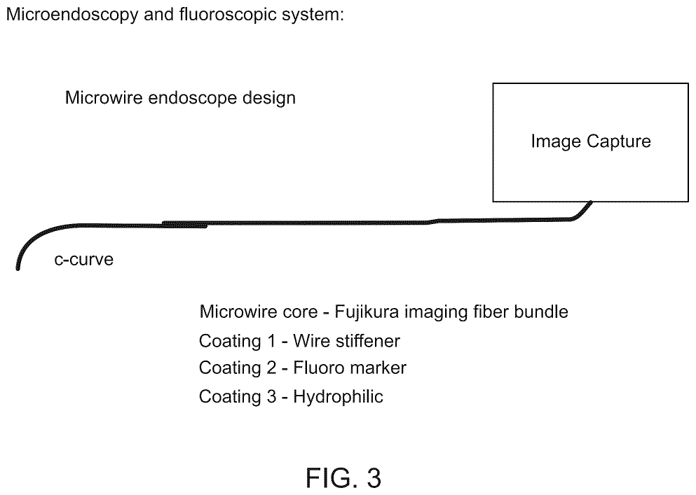

FIG. 3 continues the illustrative but not limiting cartooned schematic depiction of the microendoscopy and fluoroscopic system, including the proferred microwire endoscope design which further comprises a microwire core of such as an fiber imaging bundle (Fujikura, Japan), wire stiffener, fluoro marker and hydrophilic coating. Artisans shall understand that the c-curve and image capture depict functional elements of the present invention as shown and claimed below.

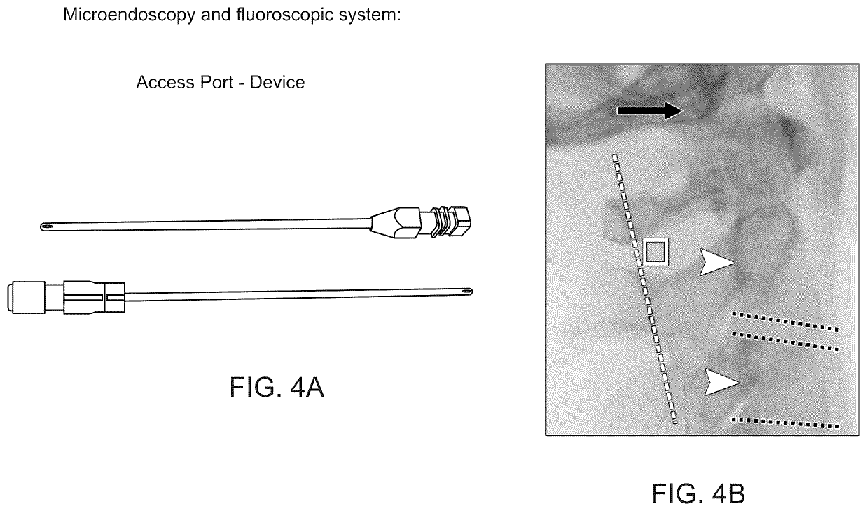

FIG. 4A shows the needle device and access port being a guide for directing catheter cephalad as it exists access needle, as known to those skilled in the art, namely interventional radiologists, brain surgeons and related clinicians.

FIG. 4B is a radiographic image showing the precise location for the access needle/port, once again, known to those of skill in the art.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary. Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A new process to treat hydrocephalus, absent craniotomy, which comprises, in combination:
providing a microcatheter-based endoscopy system, further comprising at least a cerebral aqueduct stent and balloon, being emplaceable via a specialized needle-based device and access port as a guide for directing catheter cephalad as it exits the access needle.

2. The process of claim 1, with the ability to be used concurrently with fluoroscopy.

3. The process of claim 2, using the subject imaging to define precise emplacement including the use of radiopaque markers to avoid brainstem impacts safely.

4. A minimally invasive system for accessing the ventricular spaces and aqueducts within the brain, which comprises, in combination:
at least a beveled needle long enough to span from the outside of the skull into ventricular territory;
a microcatheter and wire(s) set to support the access path created by the at least a needle;
at least a stenting/flow-diversion means to travel into the access path;
a fiber-optic wire means for capturing images along the access path; and,
at least a sheath for stabilizing the devices and supporting mapping ventriculography derived data, arrayed, stirred and optionally Al mediated with human intervention to create a database of patient blinded HIPAA compliant information on the procedures and devices involved.

5. A system for using minimally invasive approaches to address targets from trans-cortical brain ventricle procedures, including hydrocephalus, which comprises, in combination:
at least an ingress means further comprising a beveled needle with a cup, catheter and wires and any related medical devices and apparatus used to perform the same steps; ventrigulography-means defined by at least a fiber-optic wire out cable having catheter based access, delivery and travel means; wherein angles between sixty and ninety degrees permit ingress to ventricular spaces outside of the risk of brainstem contact; and
stenting or flow-vertex or thrombectomy means to cure cerebrospinal flow defect issues.

6. A process for addressing hydrocephalus and related brain ventricular space issues; comprising, in combination:
providing a bevelled needle with cup means for accessing the target zones via creating an aperture;
inserting catheters and wires, including imaging, to confirm and address subject cerebrospinal fluid or fluid flow conditions needing amelioration; and
confirming resultory ameliorative interventions or repeating other therapies.

7. Products by the process of claim 6, above and being other and further procedures to address hydrocephalus and related issues.

8. Products by the process of claim 6, above being medical devices emplaced within the ventricular spaces, aqueducts or formina.

9. A novel enhanced methodology for replacing craniotomy, comprising:
a percutaneous approach to the perimedullary and/or perispinalcord space using image guidance being at least more than one of fluoroscopy, CT, MRI and stereotactic approaches.

10. The novel enhanced methodology of claim 9, using a needle and/or wire to create a sheath or tracer access port, including advancement of a catheter and/or treatment device in either cephalad, caudal or another direction, such as the spinal canal.

11. The novel enhanced methodology of claim 10, including ventriculography by contrast infusion through the access port or through catheters positioned at other locations within the ventricular or thecal space, using status or dynamic versions; and/or via microendoscopic evaluation.

12. The novel enhanced methodology of claim 11, further comprising pressure measurements via catheters and/or related pressure sensing devices in continuous or signal measurements; further comprising CSF active (tagged molecules fluorescent, radionuclide or contrast based) and passive measurement of metabolites, oxygen, drugs, hemoglobin, ions or other metabolic biomarkers of health or disease.

13. The novel enhanced methodology of claim 12, further comprising tissue biopsies; stenting, pasty, duroplasty, drainage, marsupialization, lysis and other structural therapies, for example stenting of the aqueduct to treat aqueductal stenosis; drainage for shunts that is single, short duration, or permanent; treating subarachnoid hemorrhage, hydrocephalus, such as where a drain is placed in the access port in the CSF space and drainage out within another peritoneal or venous space.

14. A minimally invasive cerebral ventricular access process, comprising:
(i) Access a modified Lumbar Puncture-type of (“LP”) needle and microwire using fluoroscopy; (ii) Remove LP needle and replace with endoscopy microcatheter needle port over the microwire; (iii) Measure intrathecal pressure; (iv) Instill contrast through port for ventriculography; (v) Insert microcatheter endoscopy system into the patient via the port; (vi) Direct catheter over the endoscopic microwire cephalad through the aqueduct under fluoroscopic and endoscopic visualization; (vii) Remove endoscopic microwire and measure 3rd ventricular pressure, and (viii) Perform stent placement (vs balloon angioplasty).
